(19) United States
(12) Patent Application Publication
(10) Pub. No.: US 2023/0031386 A1
(43) Pub. Date: Feb. 2, 2023

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Yasuyuki HOSONO, Kanagawa (JP); Nobuya KITAMURA, Kanagawa (JP); Kenji NAGAMIYA, Kanagawa (JP); Tomohide HIRAGAMI, Kanagawa (JP)

(21) Appl. No.: 17/811,072

(22) Filed: Jul. 7, 2022

(30) Foreign Application Priority Data
Jul. 29, 2021 (JP) 2021-124690

(51) Int. Cl. G16H 40/67

(52) U.S. Cl. CPC G16H 40/67

(57) ABSTRACT

An information processing apparatus includes at least one processor. The processor acquires input information including a text, a pictogram, an emoticon, or an image input to the information processing apparatus, or a selection item selected by using the information processing apparatus, and issues a command related to measurement or presentation of biological information to a measurement device that measures the biological information of a user, in a case in which a content of the input information satisfies a predetermined condition.

### CROSS-REFERENCE TO RELATED APPLICATIONS

The present application claims priority under 35 U.S.C. § 119 to Japanese Patent Application No., 2021-124690 filed on Jul. 29, 2021. The above application is hereby expressly incorporated by reference, in its entirety, into the present application.

### BACKGROUND

### 1. Technical Field

The disclosed technology relates to an information processing apparatus, an information processing method, and an information processing program.

### 2. Description of the Related Art

The following technologies are known as technologies of collecting information on behavior of a user and processing the information on the behavior of the user into useful information for use by the user. For example, W2012/124259A discloses an information terminal comprising a behavior specifying unit that collects behavior information of a user, a first detection unit that detects a change of the collected behavior information, and an information providing unit that provides information on behavior based on a frequency of the behavior corresponding to the detected change.

### SUMMARY

In recent years, a wearable device, such as a smart watch, has made it possible to measure biological information, such as a heartbeat, a blood pressure, and electrocardiography, of a user. In such a device, in order to suppress battery consumption, the measurement of the biological information is not always performed, but is often performed based on an instruction of the user or a predetermined schedule. It is considered preferable that the biological information be measured at an appropriate timing, for example, in a case in which an abnormality occurs in a physical condition of the user, but in a case in which the biological information is not always measured, in some cases, the measurement or presentation cannot be performed at an appropriate timing.

The disclosed technology has been made in view of the above points, and is to perform measurement or presentation of biological information at an appropriate timing.

The disclosed technology relates to an information processing apparatus comprising at least one processor. The processor acquires input information including a text, a pictogram, an emoticon, or an image input to the information processing apparatus, or a selection item selected by using the information processing apparatus, and issues a command related to measurement or presentation of biological information to a measurement device that measures the biological information of a user, in a case in which a content of the input information satisfies a predetermined condition.

The input information satisfying the condition may be information including a content related to a physical condition, a symptom, health, feeling, emotion, or a disease name of the user.

The command may be a command to start the measurement of the biological information. The command may be a command to transmit the biological information. The command may be the command to transmit the biological information at a point in time corresponding to a point in time when the input information satisfying the condition is input. The command may be a command to change a measurement condition of the biological information.

The input information may be information input on an application installed in the information processing apparatus. The input information may include a search keyword input to a search engine for searching for information on a network. The input information may include a content transmitted to or sent to a specified or unspecified party. The input information may include information on a product or service purchased or a product or service to be purchased by information communication via a network.

The processor may further issue a command related to measurement or presentation of biological information of a third party other than the user to a measurement device that measures the biological information of the third party, and is registered or authenticated in advance, in a case in which the content of the input information satisfies the condition.

The disclosed technology relates to an information processing method executed by at least one processor provided in an information processing apparatus, the method comprising acquiring, by the processor, input information including a text, a pictogram, an emoticon, or an image input to the information processing apparatus, or a selection item selected by using the information processing apparatus, and issuing, by the processor, a command related to measurement or presentation of biological information with respect to a measurement device that measures the biological information of a user, in a case in which a content of the input information satisfies a predetermined condition.

The disclosed technology relates to an information processing program causing at least one processor provided in an information processing apparatus to execute a process comprising acquiring input information including a text, a pictogram, an emoticon, or an image input to the information processing apparatus, or a selection item selected by using the information processing apparatus, and issuing a command related to measurement or presentation of biological information with respect to a measurement device that measures the biological information of a user, in a case in which a content of the input information satisfies a predetermined condition.

According to the disclosed technology, it is possible to perform the measurement or presentation of the biological information at an appropriate timing.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a biological information management system according to an embodiment of the disclosed technology;

FIG. 2 is a diagram showing an example of a hardware configuration of an information processing apparatus according to the embodiment of the disclosed technology;

FIG. 3 is a functional block diagram showing an example of a functional configuration of the information processing apparatus according to the embodiment of the disclosed technology;

FIG. 4 is a flowchart showing an example of a flow of processing performed by executing a control program according to the embodiment of the disclosed technology;

FIG. 5 is a diagram showing an example of operations of the information processing apparatus according to the embodiment of the disclosed technology;

FIG. 6 is a diagram showing an example of a configuration of a biological information management system according to another embodiment of the disclosed technology; and

FIG. 7 is a diagram showing an example of operations of the information processing apparatus according to the embodiment of the disclosed technology.

### DETAILED DESCRIPTION

In the following, one example of the embodiments of the disclosed technology will be described with reference to the drawings. It should be noted that the same or equivalent components and portions in the drawings are represented by the same reference numerals, and the overlapping description will be omitted.

### First Embodiment

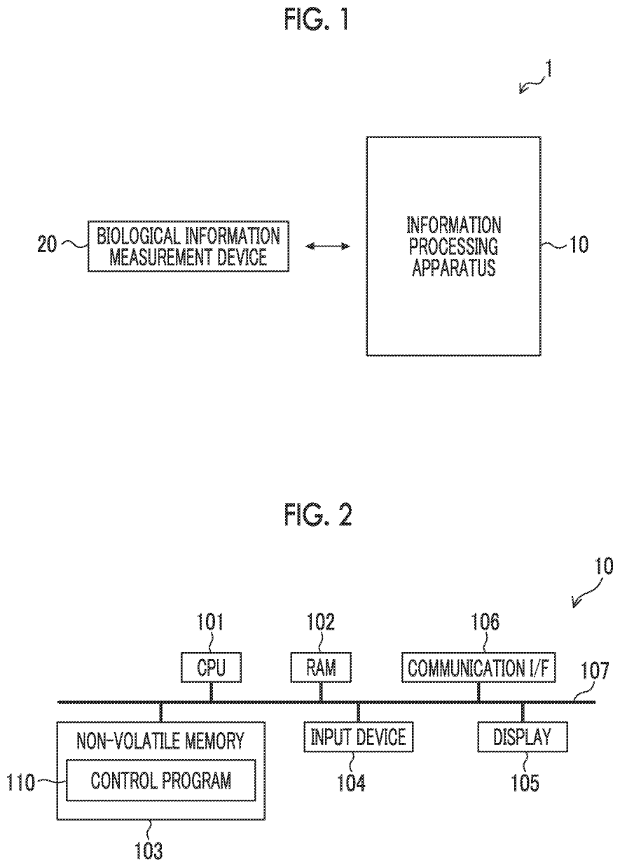

FIG. 1 is a diagram showing an example of a configuration of a biological information management system 1 according to the embodiment of the disclosed technology. A biological information management system 1 includes an information processing apparatus 10 and a biological information measurement device 20.

The information processing apparatus 10 is a communication terminal device used by a person who is a management target of the biological information (hereinafter, referred to as a user). The information processing apparatus 10 may be, for example, a desktop computer, a laptop, a tablet computer, or a smartphone.

The biological information measurement device 20 measures the biological information of the user. The biological information may be, for example, information indicating at least one of a body temperature, a heartbeat, electrocardiography, myoelectricity, a blood pressure, arterial oxygen saturation (SpO2), a blood glucose level, or a lipid level. The biological information measurement device 20 may be a wearable device, such as a smart watch, comprising a sensor that measures these pieces of the biological information. The biological information measurement device 20 measures the biological information of the user based on an operation input for performing the measurement or a command from the information processing apparatus 10. In addition, the biological information measurement device 20 can also measure the biological information in accordance with a preset schedule. The biological information measurement device 20 is communicably connected to the information processing apparatus 10, and transmits the measured biological information to the information processing apparatus 10. It should be noted that the biological information management system 1 may comprise two or more biological information measurement devices that measure different types of the biological information.

FIG. 2 is a diagram showing an example of a hardware configuration of the information processing apparatus 10. The information processing apparatus 10 includes a central processing unit (CPU) 101, a random access memory (RAM) 102, a non-volatile memory 103, a display 105, an input device 104 including a keyboard, a mouse, and the like, and a communication interface 106. The hardware is connected to a bus 107.

The communication interface 106 is an interface for the information processing apparatus 10 to perform communication with the biological information measurement device 20. A communication method may be any of wired or wireless. For wireless communication, for example, a method compliant with an existing wireless communication standard, such as Wi-Fi (registered trademark) and Bluetooth (registered trademark), can be applied. The non-volatile memory 103 is a storage medium, such as a hard disk and a flash memory, and stores a control program 110 described below. The RAM 102 is a work memory for the CPU 101 to execute processing. The CPU 101 loads the control program 110 stored in the non-volatile memory 103 into the RAM 102, and executes the processing in accordance with the control program 110. The CPU 101 is an example of a “processor” according to the disclosed technology.

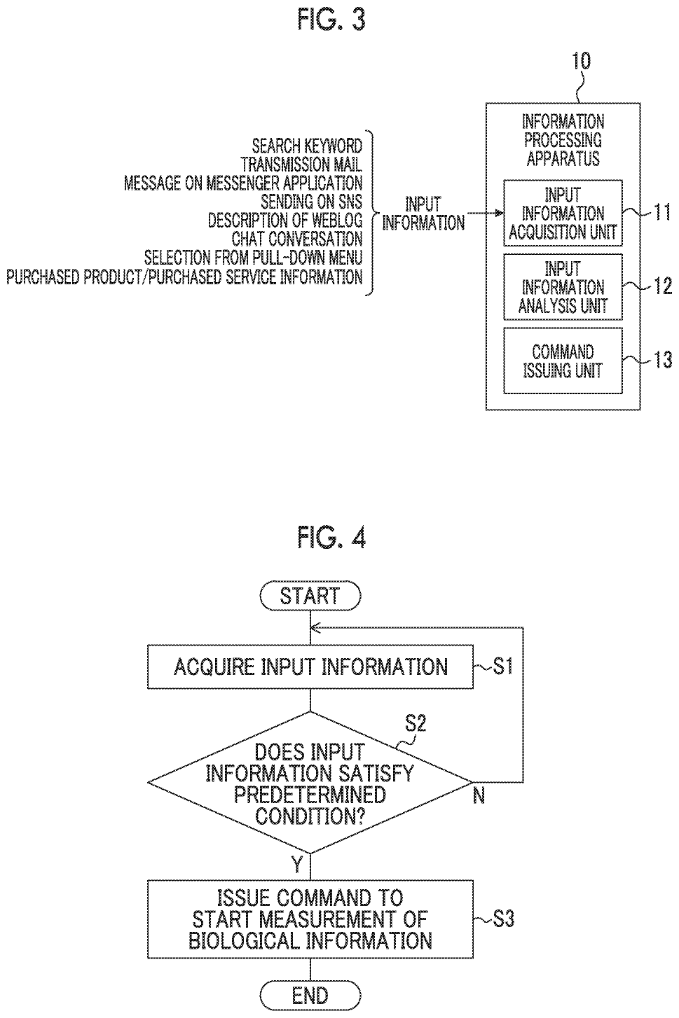

FIG. 3 is a functional block diagram showing an example of a functional configuration of the information processing apparatus 10. The information processing apparatus 10 includes an input information acquisition unit 11, an input information analysis unit 12, and a command issuing unit 13. By the CPU 101 executing the control program 110, the CPU 101 functions as the input information acquisition unit 11, the input information analysis unit 12, and the command issuing unit 13.

The input information acquisition unit 11 acquires input information input to the information processing apparatus 10. The input information can include a text, a pictogram, an emoticon, or an image input to the information processing apparatus 10 or a selection item selected by using the information processing apparatus 10. The input information may be information input on various applications installed in the information processing apparatus 10.

The text as the input information may be, for example, a search keyword input to a search engine for searching for information on a network. In addition, the text as the input information may be a text transmitted or sent to a specified or unspecified party, such as a transmission mail, a message on a messenger application, sending on social networking service (SNS), description of a weblog, or a chat conversation. The selection item as the input information may be an item selected by using a graphical user interface (GUI) for selecting from a plurality of options, such as a pull-down menu or a radio button. In addition, the input information may be information (for example, a category of the product or service, a product name, or a service name) on a product or service purchased or a product or service to be purchased from an electronic commerce (EC) site by information communication via the network by using a web browser installed in the information processing apparatus 10. It should be noted that the input information acquisition unit 11 may use the product or service viewed for the purchase of the product or service on the EC site as the product or service to be purchased, and may acquire the information on the product or service as the input information. The input information acquisition unit 11 may acquire a combination of any two or more of information described above as the input information.

The input information acquisition unit 11 acquires the input information by linking with various applications, such as the web browser, an e-mail application, a messenger application, and an SNS application installed in the information processing apparatus 10. The input information acquisition unit 11 provides the input date and time information indicating the date and time when the input information is input to the information processing apparatus 10 to the input information and stores the input information in the non-volatile memory 103.

The input information analysis unit 12 analyzes the input information acquired by the input information acquisition unit 11 to determine whether or not the input information satisfies a predetermined condition. Specifically, in a case in which the acquired input information includes a content related to a physical condition, a symptom, health, feeling, emotion, or a disease name of the user, the input information analysis unit 12 determines that the input information satisfies the predetermined condition.

In a case in which the word, the pictogram, the emoticon, or the image registered in advance as the content related to the physical condition, the symptom, the health, the feeling, the emotion, or the disease name of the user is included in the text, the pictogram, the emoticon, the image, or the selection item as the input information, the input information analysis unit 12 may determine that the input information satisfies the predetermined condition. For example, in a case in which the input information is the search keyword input to the search engine, in a case in which the word registered in advance, such as “fever”, “cough”, “headache”, “stomach ache”, “dizzy”, “cold”, “cannot sleep”, “anxiety”, “feel depressed”, “feel listless”, “get annoyed”, “influenza”, “novel coronavirus”, or “COVID-19”, is included in the search keyword, the input information analysis unit 12 may determine that the search keyword is the input information satisfying the predetermined condition. In addition, for example, in a case in which the input information is the message input on the messenger application, in a case in which the registered word described above, or the pictogram, the emoticon, or the image (stamp) having the same meaning is included in the message, the input information analysis unit 12 may determine that the message is the input information satisfying the predetermined condition. The word related to the physical condition, the symptom, the health, the feeling, the emotion, or the disease name of the user may include onomatopoeia, such as “weep”, “disgusted”, or “pit-a-pat”.

In addition, in a case in which the input information is the information on the product purchased or the product to be purchased by the information communication via the network, in a case in which the product is a medical product, such as medicine, the input information analysis unit 12 may specify the information on the product as the input information satisfying the predetermined condition. In addition, in a case in which the input information is the information on the service purchased or the service to be purchased by the information communication via the network, in a case in which the service is related to health service or medical service aimed at maintaining, promoting, or recovering of the health, or eliminating, ameliorating, or alleviating the symptom, the input information analysis unit 12 may specify the input information including the information on the service as the input information satisfying the predetermined condition.

In a case in which the input information acquired by the input information acquisition unit 11 is determined by the input information analysis unit 12 to satisfy the predetermined condition, the command issuing unit 13 issues the command to start the measurement of the biological information of the user to the biological information measurement device 20. The biological information measurement device 20 starts the measurement of the biological information of the user in response to the command issued from the command issuing unit 13. The biological information measurement device 20 may transmit the biological information of the user measured in response to the command to the information processing apparatus 10. In this case, the information processing apparatus 10 may perform a control of displaying the biological information (measurement value) transmitted from the biological information measurement device 20 on the display 105.

FIG. 4 is a flowchart showing an example of a flow of processing performed by the CPU 101 executing the control program 110. The control program 110 is executed, for example, in a case in which the user gives an instruction to start the processing by operating the input device 104.

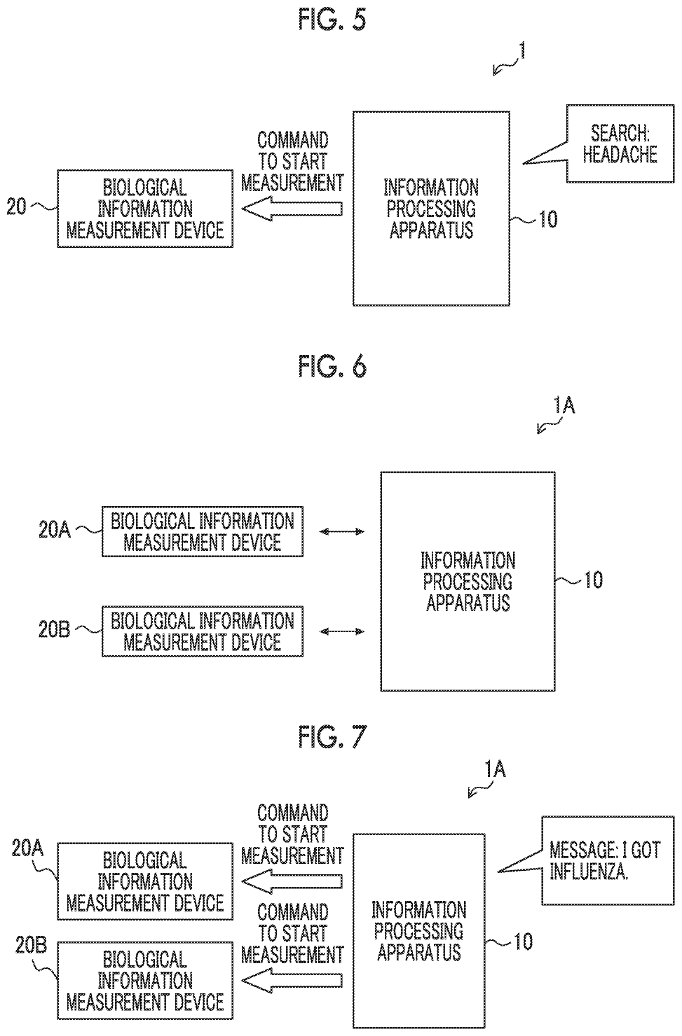

In step S1, the CPU 101 functions as the input information acquisition unit 11 to acquire the input information which is information input to the information processing apparatus 10. The input information acquisition unit 11 provides the input date and time information to the acquired input information and stores the input information in the non-volatile memory 103. FIG. 5 shows a case in which the search keyword of “headache” is input to the search engine realized on the web browser installed in the information processing apparatus 10. The input information acquisition unit 11 acquires the search keyword of “headache” as the input information.

In step S2, the CPU 101 functions as the input information analysis unit 12 to determine whether or not the input information acquired in step S1 satisfies the predetermined condition. Specifically, the input information analysis unit 12 determines whether or not the input information acquired in step S1 includes the content related to the physical condition, the symptom, the health, the feeling, the emotion, or the disease name of the user. In a case in which it is determined that the input information satisfies the predetermined condition, the processing proceeds to step S3, and in a case in which it is determined that the input information does not satisfy the predetermined condition, the processing returns to step S1. Since the search keyword of “headache” as the input information shown in FIG. 5 includes the content indicating the physical condition or the symptom of the user, the input information analysis unit 12 determines that the search keyword as the input information satisfies the predetermined condition.

In step S3, the CPU 101 functions as the command issuing unit 13 to issue the command to start the measurement of the biological information of the user to the biological information measurement device 20. FIG. 5 shows a case in which the command to start the measurement of the biological information of the user is issued to the biological information measurement device 20 in accordance with the search keyword of “headache” as the input information.

The biological information measurement device 20 starts the measurement of the biological information of the user in response to the command issued from the command issuing unit 13. The biological information measurement device 20 may transmit the biological information of the user measured in response to the command to the information processing apparatus 10. In this case, the information processing apparatus 10 may perform the control of acquiring the biological information transmitted from the biological information measurement device 20 and displaying the acquired biological information on the display 105.

As described above, the information processing apparatus 10 according to the embodiment of the disclosed technology acquires the input information including the text, the pictogram, the emoticon, or the image input to the information processing apparatus, or the selection item selected by using the information processing apparatus, and issues the command to start the measurement of the biological information to the measurement device that measures the biological information of the user, in a case in which the content of the input information satisfies the predetermined condition. The input information satisfying the predetermined condition is the information including the content related to the physical condition, the symptom, the health, the feeling, the emotion, or the disease name of the user. The input information can be information input on an application installed in the information processing apparatus 10.

For example, in a case in which the user is aware of an abnormality in the physical condition, it is assumed that the user performs a web search using a word indicating the subjective symptom as the search keyword, complains the subjective symptom to the specified or unspecified party via the messenger application and the SNS, or purchases the medicine in accordance with the subjective symptom. With the information processing apparatus 10 according to the present embodiment, since the command to start the measurement of the biological information is issued to the biological information measurement device 20 in accordance with the input information input in accordance with the subjective symptom of the user, even in a case in which the measurement of the biological information is not always performed, it is possible to obtain the biological information at an appropriate timing, such as a case in which an abnormality occurs in the physical condition of the user. In addition, with the information processing apparatus according to the present embodiment, since the measurement of the biological information is started in accordance with the input information, even in a case in which a specific operation for starting the measurement of the biological information of the user is not performed, it is possible to start the measurement of the biological information. That is, the user can be unconscious about a measurement timing of the biological information.

### Second Embodiment

In the information processing apparatus 10 according to the first embodiment, the command issuing unit 13 issues the command to start the measurement of the biological information to the biological information measurement device 20. On the other hand, in the information processing apparatus 10 according to the second embodiment of the disclosed technology, the command issuing unit 13 issues a command to transmit the biological information to the biological information measurement device 20.

Hereinafter, with reference to a specific example, an aspect will be described in which the information processing apparatus 10 issues the command to transmit the biological information, but the disclosed technology is not limited to this aspect. Here, the biological information measurement device 20 measures the body temperature as the biological information in accordance with the preset time schedule, and stores the measurement value in an internal memory (not shown) provided in the biological information measurement device 20. For example, the biological information measurement device 20 measures the body temperature of the user at 8:00, 12:00, 16:00, and 20:00, and stores the measurement values in the internal memory.

In a case in which the input information acquired by the input information acquisition unit 11 is determined by the input information analysis unit 12 to satisfy the predetermined condition, the command issuing unit 13 issues the command to transmit the measurement values of the body temperature to the biological information measurement device 20. The biological information measurement device 20 transmits the most recent measurement value among the measurement values of the body temperature stored in the internal memory to the information processing apparatus 10 in response to the command issued from the command issuing unit 13. It should be noted that the biological information measurement device 20 may measure the biological information in response to the transmission command issued from the command issuing unit 13, and may transmit the biological information obtained by the measurement to the information processing apparatus 10.

In addition, in a case in which a time difference (that is, a time lag) between the point in time when the input information satisfying the predetermined condition is input to the information processing apparatus 10 and a point in time when the command corresponding to the input information is issued is larger than a predetermined value, the command issuing unit 13 may issue the command to transmit the biological information at the point in time corresponding to the point in time when the input information satisfying the predetermined condition is input. The input date and time information indicating the input date and time of the input information is provided to the command. According to this aspect, even in a case in which the time lag occurs, it is possible to obtain the biological information at a point in time when the user is aware of the symptom.

The information processing apparatus 10 may perform the control of acquiring the biological information (measurement value) transmitted from the biological information measurement device 20 and displaying the acquired biological information on the display 105. It should be noted that a transmission destination of the biological information is not limited to the information processing apparatus 10, and may be, for example, an external server (not shown) that stores the biological information. The external server is connected to the information processing apparatus 10 via the network (not shown). The information processing apparatus 10 can access the external server to acquire the biological information transmitted from the biological information measurement device 20. In addition, the transmission destination of the biological information may be two or more, and in this case, the biological information measurement device 20 may include information specifying one or two or more transmission destinations in the command to transmit the biological information.

With the information processing apparatus 10 according to the present embodiment, the biological information measured by the biological information measurement device 20 can be taken into the information processing apparatus 10 at an appropriate timing, for example, in a case in which an abnormality occurs in the physical condition of the user.

### Third Embodiment

In the information processing apparatus 10 according to the first embodiment, the command issuing unit 13 issues the command to start the measurement of the biological information to the biological information measurement device 20. On the other hand, in the information processing apparatus 10 according to the third embodiment of the disclosed technology, the command issuing unit 13 issues a command to the biological information measurement device 20 to change the measurement condition in a case of measuring the biological information. The measurement condition may be, for example, a condition related to a measurement frequency of the biological information, a time of measurement, and a type of biological information to be measured. In addition, in a case in which the biological information measurement device 20 has a function of determining an abnormality in the measurement result of the biological information using a threshold value and issuing a warning in a case in which the determination is made as abnormal, a condition related to the threshold value may be included in the measurement condition.

Hereinafter, with reference to a specific example, an aspect will be described in which the information processing apparatus 10 issues the command to change the measurement condition of the biological information, but the disclosed technology is not limited to this aspect. Here, the biological information measurement device 20 measures the body temperature as the biological information of the user. In addition, in the biological information measurement device 20, the condition of “three times a day” is set as a standard measurement condition related to the measurement frequency.

In a case in which the input information acquired by the input information acquisition unit 11 is determined by the input information analysis unit 12 to satisfy the predetermined condition, the command issuing unit 13 issues the command to change the measurement condition related to the measurement frequency of the body temperature to “six times a day” to the biological information measurement device 20. The biological information measurement device 20 changes the measurement condition of the biological information in response to the command issued from the command issuing unit 13, and measures the biological information in accordance with the changed condition. The biological information measurement device 20 may transmit the biological information of the user measured in accordance with the changed condition to the information processing apparatus 10. In this case, the information processing apparatus 10 may perform a control of displaying the biological information (measurement value) transmitted from the biological information measurement device 20 on the display 105.

With the information processing apparatus 10 according to the present embodiment, for example, in a case in which an abnormality occurs in the physical condition of the user, it is possible to strengthen the monitoring of the biological information of the user.

### Fourth Embodiment

FIG. 6 is a diagram showing an example of a configuration of the biological information management system 1A according to a fourth embodiment of the disclosed technology. The biological information management system 1A includes the information processing apparatus 10 and biological information measurement devices 20A and 20B. The biological information measurement device 20A measures the biological information of the user. The biological information measurement device 20B is a device that measures the biological information of a third party (guest) other than the user, and is registered or authenticated in the information processing apparatus 10 as the device to be controlled by the information processing apparatus 10. The biological information measurement device 20B may be, for example, a wearable device worn by a young child or an elderly person, and the biological information measurement device 20A may be, for example, a wearable device worn by the user who is a guardian or a caregiver thereof

In the information processing apparatus 10 according to the present embodiment, in a case in which the input information acquired by the input information acquisition unit 11 is determined by the input information analysis unit 12 to satisfy the predetermined condition, the command issuing unit 13 issues the command to start the measurement of the biological information to both the biological information measurement devices 20A and 20B. Each of the biological information measurement devices 20A and 20B starts the measurement of the biological information of the user or the third party in response to the command issued from the command issuing unit 13. Each of the biological information measurement devices 20A and 20B may transmit the biological information of the user or the third party measured in response to the command to the information processing apparatus 10. In this case, the information processing apparatus 10 may perform a control of displaying the biological information (measurement value) transmitted from the biological information measurement devices 20A and 20B on the display 105.

FIG. 7 shows a case in which a text of “get influenza” is input on the messenger application installed in the information processing apparatus 10. The input information acquisition unit 11 acquires the text described above as the input information. Since the text described above includes a content indicating the disease name of the user, the input information analysis unit 12 determines that the text is the input information satisfying the predetermined condition. In accordance with the determination, the command issuing unit 13 issues the command to start the measurement of the biological information to both the biological information measurement devices 20A and 20B.

With the information processing apparatus 10 according to the present embodiment, the measurement of biological information can be started by the third party, as well as the user in accordance with the input information input to the information processing apparatus 10. For example, in a case in which the user develops an infectious disease, there is a possibility that a close contact person, such as a family member living together, is also infected. With the information processing apparatus 10 according to the present embodiment, by setting the third party who can be the close contact person as the measurement target by the biological information measurement device 20B, in a case in which an abnormality occurs in the physical condition of the user, it is possible to quickly start the measurement of the biological information by the third party, as well as the user.

It should be noted that the command issuing unit 13 may issue the command to transmit the biological information to both the biological information measurement devices 20A and 20B. In addition, the command to change the measurement condition in a case of measuring the biological information may be issued to both the biological information measurement devices 20A and 20B.

In the embodiments described above, as a hardware structure of the processing units that execute various pieces of processing, such as the input information acquisition unit 11, the input information analysis unit 12, and the command issuing unit 13, the following various processors can be used. The various processors include, for example, a programmable logic device (PLD), such as an FPGA, which is a processor of which a circuit configuration can be changed after manufacture, and a dedicated electrical circuit, such as an application specific integrated circuit (ASIC), which is a processor having a dedicated circuit configuration designed to execute specific processing, in addition to the CPU and the GPU which are general-purpose processors that execute software (program) to function as various processing units as described above.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

A first example of the configuration in which the plurality of processing units are composed of one processor is a form in which one processor is composed of a combination of one or more CPUs and software and the processor functions as the plurality of processing units, as represented by the computer, such as a client and a server. A second example thereof is a form in which a processor that realizes all of the functions of a system including the plurality of processing units with one integrated circuit (IC) chip is used, as represented by a system-on-chip (SoC). In this way, various processing units are composed of one or more of the various processors as the hardware structure.

Further, more specifically, an electric circuit (circuitry) obtained by combining circuit elements, such as semiconductor elements, can be used as the hardware structure of the various processors.

In addition, in the embodiments described above, the aspect has been described in which the control program 110 is stored (installed) in advance in the non-volatile memory 103, but the disclosed technology is not limited to this. The control program 110 may be provided in a form being recorded on a recording medium, such as a compact disc read only memory (CD-ROM), a digital versatile disc read only memory (DVD-ROM), or a universal serial bus (USB) memory. In addition, the control program 110 may have a form being downloaded from the external device via the network. Explanation of References

What is claimed is:

1. An information processing apparatus comprising:
at least one processor,
wherein the processor
acquires input information including a text, a pictogram, an emoticon, or an image input to the information processing apparatus, or a selection item selected by using the information processing apparatus, and
issues a command related to measurement or presentation of biological information to a measurement device that measures the biological information of a user, in a case in which a content of the input information satisfies a predetermined condition.

2. The information processing apparatus according to claim 1,
wherein the input information satisfying the condition is information including a content related to a physical condition, a symptom, health, feeling, emotion, or a disease name of the user.

3. The information processing apparatus according to claim 1,
wherein the command is a command to start the measurement of the biological information.

4. The information processing apparatus according to claim 1,
wherein the command is a command to transmit the biological information.

5. The information processing apparatus according to claim 4,
wherein the command is the command to transmit the biological information at a point in time corresponding to a point in time when the input information satisfying the condition is input.

6. The information processing apparatus according to claim 1,
wherein the command is a command to change a measurement condition of the biological information.

7. The information processing apparatus according to claim 1,
wherein the input information is information input on an application installed in the information processing apparatus.

8. The information processing apparatus according to claims 1,
wherein the input information includes a search keyword input to a search engine for searching for information on a network.

9. The information processing apparatus according to claim 1,
wherein the input information includes a content transmitted to or sent to a specified or unspecified party.

10. The information processing apparatus according to claim 1,
wherein the input information includes information on a product or service purchased or a product or service to be purchased by information communication via a network.

11. The information processing apparatus according to claim 1,
wherein the processor further issues a command related to measurement or presentation of biological information of a third party other than the user to a measurement device that measures the biological information of the third party, and is registered or authenticated in advance, in a case in which the content of the input information satisfies the condition.

12. An information processing method executed by at least one processor provided in an information processing apparatus, the method comprising:
acquiring, by the processor, input information including a text, a pictogram, an emoticon, or an image input to the information processing apparatus, or a selection item selected by using the information processing apparatus; and
issuing, by the processor, a command related to measurement or presentation of biological information with respect to a measurement device that measures the biological information of a user, in a case in which a content of the input information satisfies a predetermined condition.

13. A non-transitory computer-readable storage medium storing an information processing program causing at least one processor provided in an information processing apparatus to execute a process comprising:
acquiring input information including a text, a pictogram, an emoticon, or an image input to the information processing apparatus, or a selection item selected by using the information processing apparatus; and
issuing a command related to measurement or presentation of biological information with respect to a measurement device that measures the biological information of a user, in a case in which a content of the input information satisfies a predetermined condition.
